(19) United States
(12) Patent Application Publication
(10) Pub. No.: US 2020/0072806 A1
(43) Pub. Date: Mar. 5, 2020

(54) ENVIRONMENTAL SENSOR WITH INTEGRATED ARITHMETICAL PROCESSING CORE

(71) Applicant: STMICROELECTRONICS S.r.l., Agrate Brianza (IT)

(72) Inventors: Marco LEO, Locate di Triulzi (IT); Massimo Michele Antonio SORBERA, Valverde (IT)

(21) Appl. No.: 16/116,568

(22) Filed: Aug. 29, 2018

(51) Int. Cl. G01N 33/00; G01N 27/12

(52) U.S. Cl. CPC G01N 33/0009; G01N 27/128; G01N 27/123

(57) ABSTRACT

An integrated device on a chip includes an environmental sensor, one or more processing cores coupled to the environmental sensor, and a memory coupled to the one or more processing cores. An interface is coupled to the one or more processing cores. At least one of the processing cores includes an arithmetical floating-point circuit. In operation, the arithmetical floating-point circuit executes instructions to process sensor data received from the environmental sensor by the one or more processing cores. The processing core calculates environmental results information based on the processed sensor data, and provides the calculated environmental results information via the interface.

### BACKGROUND

### Technical Field

The following disclosure relates generally to low-power sensor systems, and in particular to environmental sensor systems.

### Description of the Related Art

Environmental sensors are associated with high levels of arithmetic computation in order to derive calculated results information based on raw physical sensor data, as well as to compensate for changes in external factors. For example, gas sensor results data regarding a particular gas concentration may need to be recalculated when external factors such as temperature and humidity fluctuate in order to provide accurate results for the gas concentration being measured.

Prior solutions to performing such arithmetic computation have involved providing sensor data to one or more external application processors in order to implement environmental sensors using low power requirements and low complexity. However, such solutions require the addition of an additional component to many systems that would not otherwise require an external application processor, and may also undesirably increase power consumption and complexity in the overall system.

### BRIEF SUMMARY

In an embodiment, an integrated device comprises environmental sensing circuitry integrated into a chip, which, in operation, generates environmental sensor data related to a first environmental condition; a processing core integrated into the chip and coupled to the environmental sensing circuitry, the processing core including an arithmetical floating-point circuit; and an interface coupled to the processing core. In operation, the processing core generates environmental results information related to the first environmental condition based on at least some of the environmental sensor data generated by the environmental sensing circuitry and controls transmission of environmental results information via the interface. In an embodiment, the integrated device comprises a memory, which, in operation stores instructions for execution by the processing core, and one or more non-volatile memories. In an embodiment, in operation, the one or more non-volatile memories store at least some of environmental sensor data generated by the environmental sensor. In an embodiment, in operation, the one or more non-volatile memories store multiple instances of the at least some environmental sensor data, and the processing core validates the at least some environmental sensor data by performing a comparison between the stored multiple instances of the at least some environmental sensor data. In an embodiment, in operation, the one or more non-volatile memories store at least some of environmental results information generated by the processing core. In an embodiment, in operation, the one or more non-volatile memories store multiple instances of the at least some environmental results information, and the processing core validates the at least some environmental results information by performing a comparison between the stored multiple instances of the at least environmental results information. In an embodiment, the integrated device comprises one or more random-access memories. In an embodiment, generating the environmental results information includes applying compensation based on one or more environmental factors. In an embodiment, the one or more environmental factors include at least one of a group that includes temperature, pressure, illumination, sensor location, sensor position, sensor orientation, and long-term sensor drift. In an embodiment, the compensation is based on changes in one or more of the environmental factors. In an embodiment, the environmental sensing circuitry comprises a gas sensor. In an embodiment, the environmental sensing circuitry includes one or more of a group that includes a humidity sensor, a chemical sensor, a biochemical compound sensor, a radioisotope sensor, an infrared sensor, an air quality sensor, a water quality sensor, a thermal sensor, and an organic compound detector. In an embodiment, the environmental sensing circuitry, in operation, generates environmental sensor data related to a second environmental condition, and the processing core, in operation, generates the environmental results information related to the first environmental condition based on at least some of the environmental sensor data related to the first environmental condition and at least some of the environmental sensor data related to the second environmental condition. In an embodiment, the first environmental condition is an indication of a presence of a gas and the second environmental condition is an indication of a temperature.

In an embodiment, a system comprises: an application processor; one or more memories coupled to the application processor; and an integrated sensor device integrated into a chip, which includes: environmental sensing circuitry, which, in operation, generates environmental sensor data related to a first environmental condition; a processing core coupled to the environmental sensing circuitry and including an arithmetical floating-point circuit; and an interface coupled to the processing core. In operation, the processing core: generates environmental results information related to the first environmental condition based on at least some of the environmental sensor data generated by the environmental sensing circuitry; and controls transmission of environmental results information to the application processor via the interface. In an embodiment, the integrated sensor device comprises one or more non-volatile memories, which, in operation, store at least some environmental results information generated by the processing core. In an embodiment, generating the environmental results information includes applying compensation based on one or more environmental factors. In an embodiment, the one or more environmental factors include at least one of a group that includes temperature, pressure, illumination, sensor location, sensor position, sensor orientation, and long-term sensor drift. In an embodiment, the environmental sensing circuitry includes one or more of a group that includes a humidity sensor, a chemical sensor, a biochemical compound sensor, a radioisotope sensor, an infrared sensor, an air quality sensor, a water quality sensor, a thermal sensor, and an organic compound detector. In an embodiment, the environmental sensing circuitry, in operation, generates environmental sensor data related to a second environmental condition; and the processing core, in operation, generates the environmental results information related to the first environmental condition based on at least some of the environmental sensor data related to the first environmental condition and at least some of the environmental sensor data related to the second environmental condition.

In an embodiment, a method comprises: generating, using an environmental sensing circuitry of an integrated sensing device, environmental sensor data related to a first environmental condition; generating, using a processing core of the integrated sensing device, the processing core including an arithmetical floating point circuit, environmental results information based on at least some of the environmental sensor data related to the first environmental condition generated using the environmental sensing circuitry; and transmitting, using an interface of the integrated sensing device and under control of the processing core, the generated environmental results information. In an embodiment, the method comprises: storing, in a memory of the integrated sensing device, instructions to control generating of the environmental results information; and executing, by the processing core, of the stored instructions. In an embodiment, the method comprises: storing, in a non-volatile memory of the integrated sensing device, at least some of the environmental results information generated by the processing core. In an embodiment, the method comprises: generating environmental sensor data related to a second environmental condition, wherein the generating the environmental results information related to the first environmental condition is based on at least some of the environmental sensor data related to the first environmental condition and at least some of the environmental sensor data related to the second environmental condition.

In an embodiment, a non-transitory computer-readable medium stores instructions which, when executed by a processing core of an integrated sensing device including an arithmetic floating-point circuit, cause the integrated sensing device to perform a method. The method comprises generating environmental sensor data related to a first environmental condition; generating environmental results information based on at least some of the environmental sensor data related to the first environmental condition generated using the environmental sensing circuitry; and controlling transmission of the generated environmental results information. In an embodiment, the method comprises: storing, in a non-volatile memory of the integrated sensing device, at least some of the generated environmental results information. In an embodiment, the method comprises: generating environmental sensor data related to a second environmental condition, wherein the generating the environmental results information related to the first environmental condition is based on at least some of the environmental sensor data related to the first environmental condition and at least some of the environmental sensor data related to the second environmental condition.

In an embodiment, an integrated device comprises: environmental sensing circuitry, which, in operation, generates environmental sensor data related to a plurality of environmental conditions; a processing core coupled to the environmental sensing circuitry; an interface coupled to the processing core, wherein, in operation, the processing core: generates environmental results information as a function of the environmental sensor data related to the plurality of environmental conditions; and controls transmission of environmental results information via the interface. In an embodiment, the integrated device comprises one or more non-volatile memories, which, in operation store at least some of the environmental results information. In an embodiment, in operation, the one or more non-volatile memories store multiple instances of the at least some environmental results information and the processing core validates the at least some environmental sensor data by performing a comparison between the stored multiple instances of the at least some environmental results information. In an embodiment, the environmental results information comprises an indication of a concentration of a gas and the plurality of environmental conditions comprise one or more of temperature, pressure, illumination, sensor location, sensor position, sensor orientation, and long-term sensor drift. In an embodiment, the environmental sensing circuitry comprises a gas sensor.

In an embodiment, a system comprises: an application processor; one or more memories coupled to the application processor; and an integrated device, comprising: environmental sensing circuitry, which, in operation, generates environmental sensor data related to a plurality of environmental conditions; a processing core coupled to the environmental sensing circuitry; and an interface coupled to the processing core, wherein, in operation, the processing core: generates environmental results information as a function of the environmental sensor data related to the plurality of environmental conditions; and controls transmission of environmental results information to the application processor via the interface. In an embodiment, the integrated sensor device comprises one or more non-volatile memories, which, in operation, store at least some environmental results information generated by the processing core. In an embodiment, the environmental sensing circuitry includes one or more of a group that includes a humidity sensor, a chemical sensor, a biochemical compound sensor, a radioisotope sensor, an infrared sensor, an air quality sensor, a water quality sensor, a thermal sensor, and an organic compound detector.

In an embodiment, a method comprises: generating, using environmental sensing circuitry of an integrated sensor, environmental sensor data related to a plurality of environmental conditions; generating, using a processing core of the integrated sensor, environmental results information as a function of the environmental sensor data related to the plurality of environmental conditions; and transmitting, under control of the processing core, environmental results information via an interface of the integrated sensor. In an embodiment, the method comprises storing at least some of the environmental results information in a non-volatile memory of the integrated sensor. In an embodiment, the environmental results information comprises an indication of a concentration of a gas and the plurality of environmental conditions comprise one or more of temperature, pressure, illumination, sensor location, sensor position, sensor orientation, and long-term sensor drift.

In an embodiment, an environmental sensor comprises: means for sensing environmental data related to a plurality of environmental conditions; means for generating environmental results information as a function of sensed environmental data related to the plurality of environmental conditions; and means for transmitting environmental results information generated by the means for generating. In an embodiment, the environmental results information comprises an indication of a concentration of a gas and the plurality of environmental conditions comprise one or more of temperature, pressure, illumination, sensor location, sensor position, sensor orientation, and long-term sensor drift.

### BRIEF DESCRIPTION OF THE SEVERAL VIEWS OF THE DRAWINGS

FIG. 1A is a functional block diagram of an environmental sensor system.

FIG. 1B is a functional block diagram of an embodiment of an environmental sensor system in accordance with various techniques presented herein.

FIGS. 2A-2B are functional block diagrams of embodiments of environmental sensor systems in accordance with various techniques presented herein.

FIG. 3 illustrates a non-volatile memory storage specification that may be used in accordance with various techniques presented herein.

FIG. 4 illustrates an embodiment of a method of operating an integrated sensor device.

FIG. 5 illustrates an embodiment of a method of operating an integrated sensor device.

### DETAILED DESCRIPTION

In the following description, certain details are set forth in order to provide a thorough understanding of various embodiments of devices, systems, methods and articles. However, one of skill in the art will understand that other embodiments may be practiced without these details. In other instances, well-known structures and methods associated with, for example, circuits, such as transistors, multipliers, adders, dividers, comparators, transistors, integrated circuits, logic gates, finite state machines, accelerometers, gyroscopes, magnetic field sensors, gas sensors, memories, bus systems, etc., have not been shown or described in detail in some figures to avoid unnecessarily obscuring descriptions of the embodiments.

Unless the context requires otherwise, throughout the specification and claims which follow, the word “comprise” and variations thereof, such as “comprising,” and “comprises,” are to be construed in an open, inclusive sense, that is, as “including, but not limited to.”

Reference throughout this specification to “one embodiment,” or “an embodiment” means that a particular feature, structure or characteristic described in connection with the embodiment is included in at least one embodiment. Thus, the appearances of the phrases “in one embodiment,” or “in an embodiment” in various places throughout this specification are not necessarily referring to the same embodiment, or to all embodiments. Furthermore, the particular features, structures, or characteristics may be combined in any suitable manner in one or more embodiments to obtain further embodiments.

The headings are provided for convenience only, and do not interpret the scope or meaning of this disclosure.

The sizes and relative positions of elements in the drawings are not necessarily drawn to scale. For example, the shapes of various elements and angles are not drawn to scale, and some of these elements are enlarged and positioned to improve drawing legibility. Further, the particular shapes of the elements as drawn are not necessarily intended to convey any information regarding the actual shape of particular elements, and have been selected solely for ease of recognition in the drawings.

The present disclosure is directed to techniques for providing generated environmental results information directly from an integrated sensor device (such as a system-on-chip or “SoC” device), without requiring raw physical data from an environmental sensor to be separately processed by an external application processor in order to generate environmental results information. For example, rather than providing raw physical sensor data to an external application processor for processing, in various embodiments an integrated environmental sensor device on a chip may include one or more processing cores to perform such processing, and one or more interfaces via which the calculated/generated environmental results information may be provided. In at least some embodiments, at least one of the one or more processing cores included within the integrated environmental sensor device may include an arithmetical floating-point circuit, such as an arithmetic co-processing circuit. In this manner, the integrated environmental sensor device may provide similar information as a system that includes a non-integrated sensor device communicatively coupled to an external application processor, while additionally providing one or more benefits such as reduced power consumption, reduced silicon area, reduced ASIC complexity, and increased flexibility.

As used herein, “environmental sensor” may refer to any component that generates electrical and/or electronic signals indicative of one or more substances and/or conditions present in its immediate surroundings, and may include as non-limiting examples: a humidity sensor, particle detector, gas sensor, chemical sensor, biochemical sensor, radioisotope sensor, infrared sensor, air quality sensor, water quality sensor, thermal sensor, organic compound detector, magnetic field sensor, pressure sensor, an accelerometer, etc., and various combinations thereof. It will be appreciated that while many examples herein are provided with respect to a “gas sensor,” any other environmental sensor may be used in accordance with the techniques presented herein.

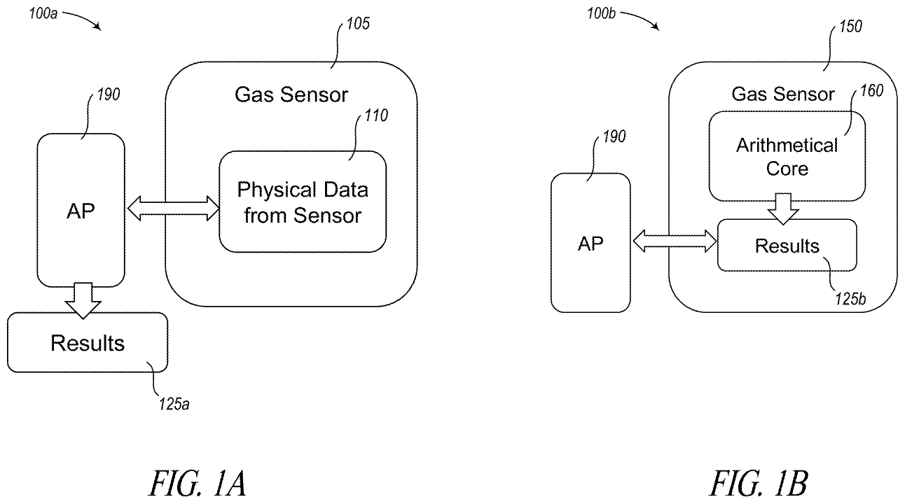

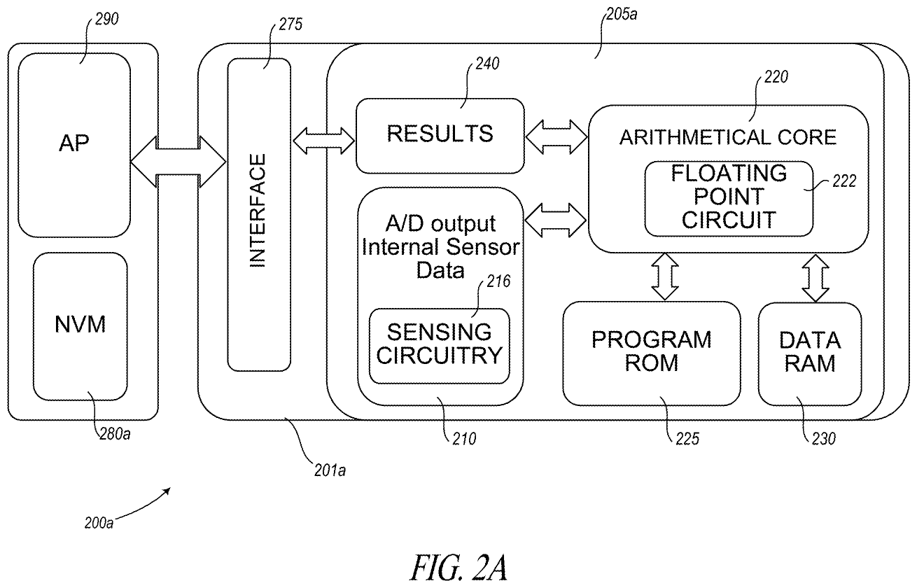

FIG. 1A depicts a functional block diagram of a known configuration for a gas sensor 105, in which raw physical data 110 from the gas sensor is provided to an external application processor 190. In the depicted known configuration, processing of the physical data from the sensor is performed by the external application processor, which in turn must devote one or more processing cycles (and corresponding power consumption) to calculating any desired environmental results information 125a based on the provided raw physical data 110 from the gas sensor 101.

FIG. 1B depicts a functional block diagram of an improved configuration for a system 101b that includes a gas sensor device 150 and integrated arithmetical processing core 160, in which raw physical data (not separately depicted) generated by the gas sensor 150 is provided directly to the arithmetical processing core 160, which calculates environmental results information 125b. In various embodiments, the integrated arithmetical processing core 160 may be embedded within the same ASIC or other package as the gas sensor 150. Because the calculated environmental results information 125b is provided directly to the external application processor 190, the external application processor is not required to devote processing cycles or power consumption prior to providing such environmental results information to external applications, client devices, or for other uses.

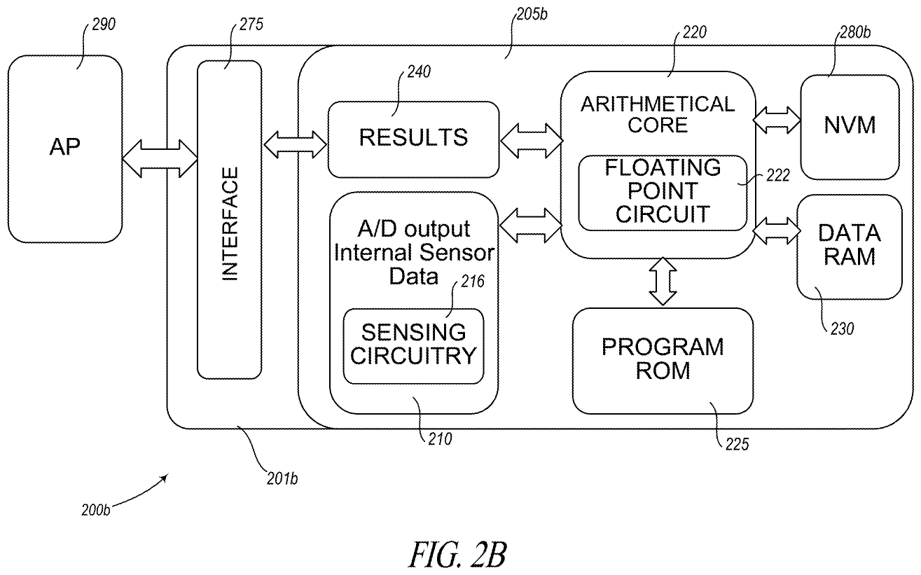

FIG. 2A depicts a functional block diagram of a sensor system 200a that includes an embodiment of an integrated sensor device or chip 201a that is communicatively coupled to an external application processor 290. In the depicted embodiment, the integrated sensor device 201a includes an environmental sensor or environmental sensing circuitry 205a that is integrally packaged on the chip 201a with and coupled to an integrated processing core 220 which includes an arithmetical floating-point circuit 222. Examples of environmental sensing circuitry that may be integrally packaged on the chip include gas sensors such as those described in U.S. Pat. No. 9,810,653 issued to Shankar et al., and U.S. Pub. No. 2012/0171713 by Cherian et al.

In at least some embodiments, the particular architecture of the integrated processing core 220 and corresponding arithmetical floating-point circuit 222 may be chosen in accordance with the anticipated underlying sensor data. As one non-limiting example, a 16-bit half-precision floating-point unit may be selected to utilize a 1-bit sign, 5-bit exponent, and 10-bit mantissa. It will be appreciated that other architectures may be utilized in accordance with the sensor type and other factors. Also in the depicted embodiment, the integrated sensor device 201a further comprises a program memory 225 (which may be read-only) and an optional random-access data memory 230, both of which are also integrated into the chip 201a and communicatively coupled to the processing core 220.

In operation, the environmental sensor 205a generates internal sensor data 210 (which may comprise analog and/or digital output data) and provides the generated internal sensor data to the processing core 220. As illustrated, the environmental sensor 205a comprises sensing circuitry 216, which may comprise one or more sensors and may sense various environmental conditions and generate sensor data. The sensor data may include various types of sensed data, such as data indicative of concentrations of gas, data indicative of pressure, data indicative of temperature, data indicative of acceleration, data indicative of device orientation, etc., and various combinations thereof.

In an embodiment, the sensing circuitry 216 comprises environmental sensing circuitry to sense temperature, integrated into the chip 201a, which, in operation, generates environmental sensor temperature data related to a first environmental condition. This raw temperature data may be compensated based on IC sensor characteristics (e.g., linear compensation: gain and offset) by the arithmetical core 220. Compensated temperature data T[deg C] may be stored in a non-volatile memory 280a in order to be used for sensing and compensating gas sensor data.

In an embodiment, the sensing circuitry 216 comprises environmental sensing circuitry to sense relative humidity, integrated into the chip 201a, which, in operation, generates environmental sensor humidity data related to a first environmental condition. The raw humidity data may be compensate as a function of temperature (e.g., compensated temperature data as discussed above) and as a function of specific coefficients, which may be stored in a non-volatile memory 280a by core 220. The relative humidity data compensated in temperature RH[%] may be stored in a non-volatile memory 280a in order to be used for sensing and compensating gas sensor data.

In an embodiment, the sensing circuitry 216 comprises environmental sensing circuitry to sense gas (the gas-sensing element may comprise a metal oxide compound deposited over a micro hot plate) which may be measured by core 220 using a sensing resistance. The value of the sensed resistance Rs may be stored in a non-volatile memory 280a.

In an embodiment, the temperature data T[deg C], the relative humidity compensated in temperature RH[%] and the sensing resistance Rs previously stored in non-volatile memory may be used to compute gas concentration in PPM and Index Air Quality (IAQ) by executing an algorithm stored in ROM 225 and using core 220 including floating point circuit 222.

Notably in contrast with the gas sensor 105 of FIG. 1A (which merely provides the raw physical data 110 from the gas sensor to an external application processor 190), the integrated processing core 220 processes the raw data. For example, the integrated processing core 220 may execute instructions stored within the program memory 225 in order to process the generated sensor data 210, and thereby calculate environmental results information 240. In the depicted embodiment, memory 225 stores executable program instructions for execution by the integrated processing core 220 in order to calculate environmental results information 240; random-access data memory 230 may be used to store various data for the processing core 220 when calculating the environmental results information, such as partial results and/or other variables. In certain embodiments, the random-access data memory 230 may be used to store one or more user-specified programs, such as to improve performance and/or to provide greater flexibility in the particular environmental results information 240 provided by the integrated sensor device 201a. As one non-limiting example with particular reference to a gas sensor, a user-specified program may be stored within the random-access data memory 230 in order to specify sequences of operations to be performed in order to detect one or more specific kinds of gas or other substance.

In various embodiments, the environmental results information 240 may comprise any number of calculated data types based on the generated sensor data 210. As non-limiting examples corresponding to an environmental gas sensor, the calculated/generated environmental results information 240 may include gas concentration data (such as may be expressed as parts-per-million or “PPM”), an Index of Air Quality (IAQ), a concentration per cubic meter (e.g., micrograms per cubic meter), etc., or other information.

In various embodiments, as part of calculating the environmental results information 240, the processing core 220 may provide short-term and/or long-term compensation for a variety of environmental factors, such as fluctuations or other changes to temperature, pressure, ambient illumination, sensor location, sensor position, sensor orientation, sensor drift or other factors, and various combinations thereof. It will be appreciated that such factors may quantitatively and/or qualitatively impact various environmental results information, such that the application of compensation for changes in such factors may allow the integrated sensor device 201a to provide more accurate environmental results information. In addition, the integrated sensor device 201a and processing core 220 may apply additional compensation based on various benchmark compensation factors, long-term drift, or other factors. Depending on the type of environmental sensor, for example, compensation factors may be attributed to lengthy durations, including multiple months or longer, and in such embodiments the integrated sensor device 201a may store at least some of the generated sensor data 210 and/or environmental results information 240 periodically (such as once per hour, once per day, or other suitable period) over the course of such duration in order to provide adequate compensation for long-term drift or other factors.

In an embodiment, one or more memories, such as the program ROM, may store contents which configure the processing core 220 to perform one or more algorithms to provide short-term and/or long-term compensation for a variety of environmental factors, such as those discussed above. The contents may be stored securely on the chip 201a, so as to facilitate preventing detection of, or unauthorized modification of, details of the one or more algorithms.

In the depicted embodiment of FIG. 2A, once the processing core 220 has calculated the environmental results information 240, it may provide the calculated environmental results information to an external application processor 290 via interface 275. In at least certain embodiments, the interface 275 may include a plurality of output registers, and as non-limiting examples may comprise an I2C (‘Inter-Integrated Circuit’) interface, SPI (‘Serial Peripheral Interface’) interface, APB (‘Advanced Peripheral Bus’) interface, or other suitable inter-circuit interface. Moreover, in at least some embodiments, the processing core 220 may additionally provide at least some of the generated sensor data 210 or other information to the external application processor 290 via interface 275, such as for storage, use by one or more external applications, or other purpose. In the depicted embodiment of FIG. 2A, the external application processor is further communicatively coupled to a non-volatile memory 280a, such as may be used to store the calculated environmental results information and/or generated sensor data.

FIG. 2B depicts a functional block diagram of a sensor system 200b that includes an embodiment of an integrated sensor device or chip 201b communicatively coupled to an external application processor 290. The integrated sensor device 201b is similar to the integrated sensor device 201a of FIG. 2A, and includes an environmental sensor 205b that is integrally packaged with and coupled to a processing core 220 that includes, for example, an arithmetical floating-point circuit 222. Also in a manner similar to the integrated sensor device 201a, the integrated sensor device 201b further comprises a program memory 225, which may be read-only, and an optional random-access data memory 230, both of which are also communicatively coupled to the processing core 220. However, in the depicted embodiment of FIG. 2B, the integrated sensor device 201b further includes an integrated non-volatile memory 280b.

In various scenarios and embodiments, the integrated non-volatile memory 280b may be coupled directly to the integrated processing core 220 (such as via a serial or parallel interface) and may be used by the integrated sensor device 201b for short- or long-term storage of generated sensor data 210 and/or calculated/generated environmental results information 240. By using non-volatile memory for such storage, the power consumption of the integrated sensor device 201b may be less than if random-access or other volatile memory is utilized for such storage.

Also in a manner similar to that described for integrated environmental sensor 205a of FIG. 2A, in operation the integrated environmental sensor 205b generates internal sensor data 210 using sensing circuitry 216 and provides the generated internal sensor data to processing core 220, which calculates environmental results information 240 using executable instructions from read-only memory 225 and then provides the calculated environmental results information to the external application processor 290 via interface 275. Some embodiments may comprise both an integrated non-volatile memory and an external non-volatile memory.

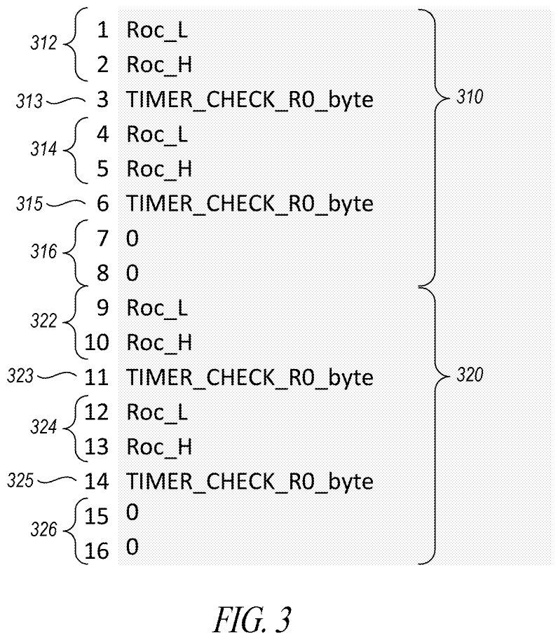

In at least some embodiments and circumstances, the integrated sensor devices 201a or 201b may validate contents of external non-volatile memory 280a or integrated non-volatile memory 280b, respectively, such as upon initiating one or more data integrity checks, upon initial power up, or other circumstances. It will be appreciated, for example, that a power interruption or shutdown of the integrated environmental sensor during a write operation may result in corruption of the contents of such non-volatile memory. In the embodiment of FIG. 3 discussed below, a solution to such corruption is provided as a redundancy check to determine whether such corruption has occurred.

FIG. 3 depicts a non-volatile memory storage specification that may be used, for example, within integrated non-volatile memory 280b. In particular, in the depicted embodiment, a 16-byte non-volatile memory register sequence is partitioned into a first 8-byte partition 310 and a second 8-byte partition 320. When data stored within the non-volatile memory is uncorrupted, each of the 8-byte partitions 310 and 320 store identical values. In particular, uncorrupted 2-byte sector 312 stores byte codes Roc_L and Roc_H, which are stored in a duplicative manner within uncorrupted 2-byte sector 322; uncorrupted bytes 313 and 315 each store byte codes TIMER_CHECK_RO_byte, which are stored in a duplicative manner within uncorrupted bytes 323 and 325; and uncorrupted 2-byte sector 316 stores null values (‘0’), which are similarly stored in a duplicative manner within uncorrupted 2-byte sector 326. As one example of a redundancy check for corruption of the non-volatile memory 280, upon power on the integrated processing core 220 may retrieve the values of each bi-partitioned memory register. In certain embodiments, only sectors with appropriately duplicated data may be considered valid by the integrated processing core 220.

It will be appreciated that in some embodiments, the depicted systems 100b, 200a and 200b, as well as integrated sensor devices 150, 201a and 201b, may include more components than illustrated, may include fewer components than illustrated, may split illustrated components into separate components, may combine illustrated components, etc., and various combinations thereof.

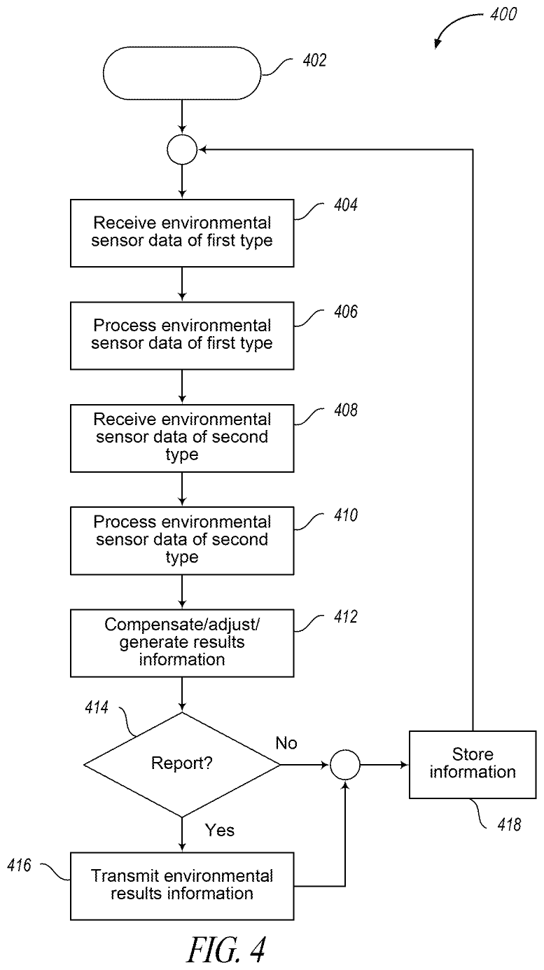

FIG. 4 illustrates an example embodiment of a method 400 of generating environmental results information based on environmental sensor data that may be employed, for example, by the environmental sensor device 205a of FIG. 2A or the environmental sensor device 205b of FIG. 2B. The method 400 starts at 402 and proceeds to 404. For convenience, the method 400 will be described with reference to FIG. 2B.

At 404, the method 400 receives environmental sensor data of a first type. For example, the sensing circuitry 216 may sense an indication of a particular gas and generate environmental sensor data indicative of the presence of the particular gas. The method 400 proceeds from 404 to 406.

At 406, the method 400 processes the environmental sensor data of the first type, generating processed environmental sensor data of a first type. For example, the processing core 220 may convert the environmental sensor data indicative of the presence of the particular gas into an indication of the concentration of the gas in parts-per-million, may generate an indication of a change in the environmental sensor data indicative of the presence of the particular gas, etc., and various combinations thereof. In some embodiments, generated environmental results information may include the processed environmental sensor data of the first type. The method 400 proceeds from 406 to 408.

At 408, the method 400 receives environmental sensor data of a second type. For example, the sensing circuitry 216 may sense an indication of a pressure and generate environmental sensor data indicative of the pressure. The method 400 proceeds from 408 to 410.

At 410, the method 400 processes the environmental sensor data of the second type, generating processed environmental sensor data of a second type. For example, the processing core 220 may convert the environmental sensor data indicative of pressure into an indication of an atmospheric pressure, may generate an indication of a change in the atmospheric pressure, etc., and various combinations thereof. In some embodiments, generated environmental results information may include the processed environmental sensor data of the second type. The method 400 proceeds from 410 to 412.

At 412, the method 400 compensates or adjusts the processed environmental sensor data of a first type based on the processed environmental sensor data of the second type, generating environmental results information. For example, the processing core 220 may adjust a concentration of a detected gas in parts-per-million generated at 406 based on a processed indication of atmospheric pressure generated at 410. In some embodiments, additional sensed indications may be processed and employed to compensate or adjust the processed environmental sensor data of the first type. For example, both a sensed indication of pressure and a sensed indication of temperature may be employed to compensate or adjust a sensed indication of a concentration of a gas to produce environmental results information related to the sensed concentration of a gas.

The method 400 proceeds from 412 to 414, where the method determines whether to report environmental results information. Reporting of environmental results information may be made periodically, in response to a request (e.g., from application processor 290), in response to generated environmental results information (e.g., information indicative of a hazard), etc., and various combinations thereof. When it is determined at 414 to report environmental results information, the method proceeds from 414 to 416, where the method 400 transmits at least some environmental results information, for example, to the application processor 290 via the interface 275 under the control of processing core 220. The method proceeds from 416 to 418. When it is not determined at 414 to report environmental results information, the method proceeds from 414 to 418.

At 418, the method 400 may store information, such as the generated environmental results information, sensed data of various types, etc. The method proceeds from 418 to 404, where additional environmental sensor data is received.

Embodiments of the method 400 may contain additional acts not shown, may omit illustrated acts, may perform acts in various orders or in parallel, etc. For example, in some embodiments additional compensation acts may be performed based, for example, on histogram information, to compensate for an age of the sensor, etc. In some embodiments, individual processing of received environmental information of various types may be omitted and raw data may be used in the generation of the results information (sensed pressure and temperature data may be used to adjust sensed gas concentration data without separate processing acts of the various types of sensed data prior to generating the results information). In some embodiments, sensed data of various types may be received and processed in parallel.

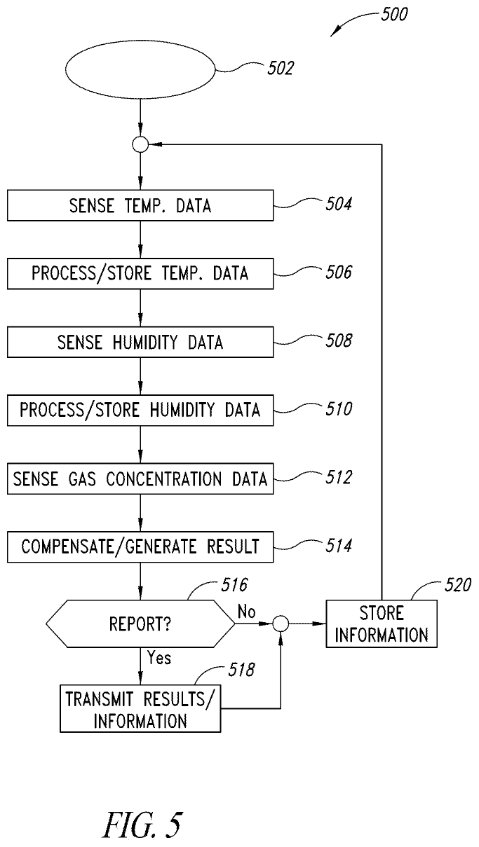

FIG. 5 illustrates an example embodiment of a method 500 of generating environmental results information based on environmental sensor data that may be employed, for example, by the environmental sensor device 205a of FIG. 2A or the environmental sensor device 205b of FIG. 2B. The method 500 starts at 502 and proceeds to 504. For convenience, the method 500 will be described with reference to FIG. 2B.

At 504, the method 500 senses raw temperature data, for example, using a temperature sensor of the sensing circuitry 216. The method 500 proceeds from 504 to 506.

At 506, the method 500 processes the raw temperature data. For example, the processing core 220 may adjust the raw temperature data based on IC sensor characteristics (e.g., linear compensation: gain and offset). The processed temperature data T[deg C] may be stored in a non-volatile memory 280b in order to be used for sensing and compensating gas sensor data. The method 500 proceeds from 506 to 508.

At 508, the method senses raw humidity data, for example, using a relative humidity sensor of the sensing circuitry 216. The method 500 proceeds from 508 to 510.

At 510, the method processes the raw humidity data. For example, the processing core 220 may adjust the raw humidity data based on temperature data (e.g., T[deg C]), adjustment coefficients (which may be stored in non-volatile memory 280b). For example, an absolute humidity HUMabs may be determined as a linear function of the relative humidity which is inversely proportional to the adjusted temperature T[deg C] according to coefficients stored in the non-volatile memory 280b. The absolute humidity may be an exponential function of the temperature T[deg C] according to a coefficient stored in the non-volatile memory 280b. The processed humidity data (e.g., HUMabs) may be stored in a non-volatile memory 280b in order to be used for sensing and compensating gas sensor data. The method 500 proceeds from 510 to 512.

At 512, the method 500 senses raw gas concentration data, for example using a gas sensor of the sensing circuitry 216 (e.g., sensing resistance Rs). The method 500 proceeds from 512 to 514.

At 514, the method 500 processes the raw gas concentration data to generate a gas concentration result. For example, the sensed resistance Rs may be compensated as a function of the adjusted temperature T[deg C], generating a value of a temperature-adjusted sensed resistance RsT, which may be an exponential function of the adjusted temperature T[deg C]. The value RsT may be adjusted based on absolute humidity HUMabs, generating a value of a hum idity-adjusted sensed resistance RsH, which may be an exponential function of the absolute humidity HUMabs. A sensitivity value S may be determined as a ratio between the adjusted sensed resistance RsH and a threshold value, such as a value corresponding to a sensed resistance in pure air which may be stored in the non-volatile memory 280b. A results value of a gas concentration in parts per million (PPM) may be determined as a function of the sensitivity value S. For example, the sensitivity value S may be compared to stored threshold values (e.g., a low-threshold sensitivity SL and a high-threshold sensitivity SH), and a function to determine a gas concentration in PPM may be selected based on the comparison. The gas concentration may depend linearly and exponentially on the sensitivity S and the absolute humidity HUMabs according to different coefficients (e.g., a first set of coefficients may be employed when S<SL, a second set of coefficients may be employed when SL<S<SH, and a third set of coefficients may be employed when S>SH). The sets of coefficients may be stored in the non-volatile memory 280b. The method 500 proceeds from 514 to 516.

At 516, the method determines whether to report the gas concentration result. Reporting of gas concentration results information may be made periodically, in response to a request (e.g., from application processor 290), in response to generated gas concentration results information (e.g., information indicative of a hazard), etc., and various combinations thereof. When it is determined at 516 to report gas concentration results information, the method proceeds from 516 to 518, where the method 500 transmits at least some environmental gas concentration results information, for example, to the application processor 290 via the interface 275 under the control of processing core 220. The method proceeds from 518 to 520. When it is not determined at 516 to report gas concentration results information, the method proceeds from 516 to 520.

At 520, the method 500 may store information, such as the generated environmental gas concentration results information, sensed data of various types, etc. The method proceeds from 520 to 504, where additional environmental sensor data is received.

Embodiments of the method 500 may contain additional acts not shown, may omit illustrated acts, may perform acts in various orders or in parallel, etc. For example, in some embodiments additional compensation acts may be performed based, for example, on histogram information, to compensate for an age of the sensor, etc. In some embodiments, individual processing of received environmental information of various types may be omitted and raw data may be used in the generation of the results information (sensed temperature data may be used to adjust sensed gas concentration data without separate processing acts of the various types of sensed temperature data prior to generating the results information). In some embodiments, sensed data of various types may be received and processed in parallel.

Details are provided below with respect to particular representations and calculations of exponential floating-point data, such as may be used by arithmetical processing core 160 of FIG. 1B and/or integrated processing core 220 of FIGS. 2A and 2B, for example, in generating environmental results information. It will be appreciated that other methods and architectures for these and other representations may be made without departing from the techniques described herein.

The formula

y=a*x^{y}

may be rewritten as

a*x^{y}=a*[e^{y*(lnx)}],

where x is a generic positive floating-point number. Such numbers may be represented as

X=2\hat{ }(exp−BIAS)*(1+(mant/(2\hat{ }NM))) where BIAS=15, NM=10

Furthermore,

$$\begin{matrix}ln ( x ) = ln [ {2}^{\hat{}} {( exp - BIAS )}^{*} ( 1 + ( mant \text{/} 2 \hat{} NM ) ) ) ] \\ = ln [ 2 \hat{} ( exp - BIAS ) ] + ln [ ( 1 + ( mant \text{/} 2 \hat{} NM ) ) ) ] \\ = {( exp - BIAS )}^{*} ln ( 2 ) + ln [ ( 1 + ( mant \text{/} ( 2 \hat{} NM ) ) ) ]\end{matrix} \\ and \\ ln ( x ) = {( exp - BIAS )}^{*} ln ( 2 ) + ln [ ( 1 + ( mant \text{/} 2 \hat{} NM ) ) ) ] ,$$

where (exp−BIAS)*ln(2) is notably a constant, and ln[(1+(mant/(2\hat{ }NM)))] can be computed with Taylor's series, as follows:

$$ln ( 1 + x ) = x - \frac{{x}^{2}}{2} + \frac{{x}^{3}}{3} - \frac{{x}^{4}}{4} + \frac{{x}^{5}}{5} - ⋯$$

Finally, e^{x} has been computed with the Taylor series:

If x>0:

$${e}^{x} = 1 + x + \frac{{x}^{2}}{2 \text{!}} + \frac{{x}^{3}}{3 \text{!}} + \frac{{x}^{4}}{4 \text{!}} + \frac{{x}^{5}}{5 \text{!}} + ⋯$$

If x<0:

$${e}^{x} = 1 - x + \frac{{x}^{2}}{2 \text{!}} - \frac{{x}^{3}}{3 \text{!}} + \frac{{x}^{4}}{4 \text{!}} - \frac{{x}^{5}}{5 \text{!}} - ⋯$$

Taylor's series may be employed in some embodiments to simplify calculations by the processing core, such as calculations used to generate results data or to apply compensation to sensor data.

An embodiment may facilitate one or more of reducing power consumption of an external application processor (e.g., application processor 190 of FIG. 1B), providing increased flexibility for compensation of raw sensor data, reduced complexity of interfaces between application processors and embedded sensors, reduced data transmission cycles between application processors and embedded sensors, etc., and various combinations thereof.

Some embodiments may take the form of or comprise computer program products. For example, according to one embodiment there is provided a computer readable medium comprising a computer program adapted to perform one or more of the methods or functions described above. The medium may be a physical storage medium, such as for example a Read Only Memory (ROM) chip, or a disk such as a Digital Versatile Disk (DVD-ROM), Compact Disk (CD-ROM), a hard disk, a memory, a network, or a portable media article to be read by an appropriate drive or via an appropriate connection, including as encoded in one or more barcodes or other related codes stored on one or more such computer-readable mediums and being readable by an appropriate reader device.

Furthermore, in some embodiments, some or all of the methods and/or functionality may be implemented or provided in other manners, such as at least partially in firmware and/or hardware, including, but not limited to, one or more application-specific integrated circuits (ASICs), digital signal processors, discrete circuitry, logic gates, standard integrated circuits, controllers (e.g., by executing appropriate instructions, and including microcontrollers and/or embedded controllers), field-programmable gate arrays (FPGAs), complex programmable logic devices (CPLDs), etc., as well as devices that employ RFID technology, and various combinations thereof.

The various embodiments described above can be combined to provide further embodiments. Aspects of the embodiments can be modified, if necessary to employ concepts of the various embodiments and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. An integrated device, comprising:
environmental sensing circuitry integrated into a chip, which, in operation, generates environmental sensor data related to a first environmental condition;
a processing core integrated into the chip and coupled to the environmental sensing circuitry, the processing core including an arithmetical floating-point circuit; and
an interface coupled to the processing core, wherein, in operation, the processing core:
generates environmental results information related to the first environmental condition based on at least some of the environmental sensor data generated by the environmental sensing circuitry; and
controls transmission of environmental results information via the interface.

2. The integrated device of claim 1, comprising:
a memory integrated into the chip, which, in operation stores instructions for execution by the processing core; and
one or more non-volatile memories integrated into the chip.

3. The integrated device of claim 2 wherein, in operation, the one or more non-volatile memories store at least some of environmental sensor data generated by the environmental sensor.

4. The integrated device of claim 3 wherein, in operation, the one or more non-volatile memories store multiple instances of the at least some environmental sensor data, and the processing core validates the at least some environmental sensor data by performing a comparison between the stored multiple instances of the at least some environmental sensor data.

5. The integrated device of claim 2 wherein, in operation, the one or more non-volatile memories store at least some of environmental results information generated by the processing core.

6. The integrated device of claim 5 wherein, in operation, the one or more non-volatile memories store multiple instances of the at least some environmental results information, and the processing core validates the at least some environmental results information by performing a comparison between the stored multiple instances of the at least environmental results information.

7. The integrated device of claim 2, comprising:
one or more random-access memories.

8. The integrated device of claim 1 wherein generating the environmental results information includes applying compensation based on one or more environmental factors.

9. The integrated device of claim 8 wherein the one or more environmental factors include at least one of a group that includes temperature, pressure, illumination, sensor location, sensor position, sensor orientation, and long-term sensor drift.

10. The integrated device of claim 8 wherein the compensation is based on changes in one or more of the environmental factors.

11. The integrated device of claim 1 wherein the environmental sensing circuitry comprises a gas sensor.

12. The integrated device of claim 1 wherein the environmental sensing circuitry includes one or more of a group that includes a humidity sensor, a chemical sensor, a biochemical compound sensor, a radioisotope sensor, an infrared sensor, an air quality sensor, a water quality sensor, a thermal sensor, and an organic compound detector.

13. The integrated device of claim 1 wherein,
the environmental sensing circuitry, in operation, generates environmental sensor data related to a second environmental condition; and
the processing core, in operation, generates the environmental results information related to the first environmental condition based on at least some of the environmental sensor data related to the first environmental condition and at least some of the environmental sensor data related to the second environmental condition.

14. The integrated device of claim 13 wherein the first environmental condition is an indication of a presence of a gas and the second environmental condition is an indication of a temperature.

15. A system, comprising:
an application processor;
one or more memories coupled to the application processor; and
an integrated sensor device which includes:
environmental sensing circuitry integrated into a chip, which, in operation, generates environmental sensor data related to a first environmental condition;
a processing core integrated into the chip and coupled to the environmental sensing circuitry, the processing core including an arithmetical floating-point circuit; and
an interface coupled to the processing core, wherein, in operation, the processing core:
generates environmental results information related to the first environmental condition based on at least some of the environmental sensor data generated by the environmental sensing circuitry; and
controls transmission of environmental results information to the application processor via the interface.

16. The system of claim 15 wherein the integrated sensor device comprises one or more non-volatile memories integrated into the chip, which, in operation, store at least some environmental results information generated by the processing core.

17. The system of claim 15 wherein generating the environmental results information includes applying compensation based on one or more environmental factors.

18. The system of claim 17 wherein the one or more environmental factors include at least one of a group that includes temperature, pressure, illumination, sensor location, sensor position, sensor orientation, and long-term sensor drift.

19. The system of claim 15 wherein the environmental sensing circuitry includes one or more of a group that includes a humidity sensor, a chemical sensor, a biochemical compound sensor, a radioisotope sensor, an infrared sensor, an air quality sensor, a water quality sensor, a thermal sensor, and an organic compound detector.

20. The system of claim 15 wherein,
the environmental sensing circuitry, in operation, generates environmental sensor data related to a second environmental condition; and
the processing core, in operation, generates the environmental results information related to the first environmental condition based on at least some of the environmental sensor data related to the first environmental condition and at least some of the environmental sensor data related to the second environmental condition.

21. A method, comprising:
generating, using an environmental sensing circuitry of an integrated sensing device on a chip, environmental sensor data related to a first environmental condition;
generating, using a processing core of the integrated sensing device, the processing core including an arithmetical floating point circuit, environmental results information based on at least some of the environmental sensor data related to the first environmental condition generated using the environmental sensing circuitry; and
transmitting, using an interface and under control of the processing core, the generated environmental results information.

22. The method of claim 21, comprising:
storing, in a memory of the integrated sensing device, instructions to control generating of the environmental results information; and
executing, by the processing core, of the stored instructions.

23. The method of claim 21, comprising:
storing, in a non-volatile memory of the integrated sensing device, at least some of the environmental results information generated by the processing core.

24. The method of claim 21, comprising:
generating environmental sensor data related to a second environmental condition, wherein the generating the environmental results information related to the first environmental condition is based on at least some of the environmental sensor data related to the first environmental condition and at least some of the environmental sensor data related to the second environmental condition.

25. A non-transitory computer-readable medium storing instructions which, when executed by a processing core of an integrated sensing device including an arithmetic floating-point circuit, cause the integrated sensing device to perform a method, the method comprising:
generating environmental sensor data related to a first environmental condition;
generating, using the processing core of the integrated sensing device, environmental results information based on at least some of the environmental sensor data related to the first environmental condition generated using the environmental sensing circuitry; and
controlling transmission of the generated environmental results information.

26. The non-transitory computer-readable medium of claim 25 wherein the method comprises:
storing, in a non-volatile memory of the integrated sensing device, at least some of the generated environmental results information.

27. The non-transitory computer-readable medium of claim 25 wherein the method comprises:
generating environmental sensor data related to a second environmental condition, wherein the generating the environmental results information related to the first environmental condition is based on at least some of the environmental sensor data related to the first environmental condition and at least some of the environmental sensor data related to the second environmental condition.

28. An integrated device, comprising:
environmental sensing circuitry integrated into a chip, which, in operation, generates environmental sensor data related to a plurality of environmental conditions;
a processing core integrated into the chip and coupled to the environmental sensing circuitry;
an interface coupled to the processing core, wherein, in operation, the processing core:
generates environmental results information as a function of the environmental sensor data related to the plurality of environmental conditions; and
controls transmission of environmental results information via the interface.

29. The integrated device of claim 28, comprising one or more non-volatile memories integrated into the chip, which, in operation store at least some of the environmental results information.

30. The integrated device of claim 29 wherein, in operation, the one or more non-volatile memories store multiple instances of the at least some environmental results information and the processing core validates the at least some environmental sensor data by performing a comparison between the stored multiple instances of the at least some environmental results information.

31. The integrated device of claim 28 wherein the environmental results information comprises an indication of a concentration of a gas and the plurality of environmental conditions comprise one or more of temperature, pressure, illumination, sensor location, sensor position, sensor orientation, and long-term sensor drift.

32. The integrated device of claim 1 wherein the environmental sensing circuitry comprises a gas sensor.

33. A system, comprising:
an application processor;
one or more memories coupled to the application processor; and
an integrated device, comprising:
environmental sensing circuitry integrated into a chip, which, in operation, generates environmental sensor data related to a plurality of environmental conditions;
a processing core integrated into the chip and coupled to the environmental sensing circuitry; and
an interface coupled to the processing core, wherein, in operation, the processing core:
generates environmental results information as a function of the environmental sensor data related to the plurality of environmental conditions; and
controls transmission of environmental results information to the application processor via the interface.

34. The system of claim 33 wherein the integrated sensor device comprises one or more non-volatile memories integrated into the chip, which, in operation, store at least some environmental results information generated by the processing core.

35. The system of claim 15 wherein the environmental sensing circuitry includes one or more of a group that includes a humidity sensor, a chemical sensor, a biochemical compound sensor, a radioisotope sensor, an infrared sensor, an air quality sensor, a water quality sensor, a thermal sensor, and an organic compound detector.

36. A method, comprising:
generating, using environmental sensing circuitry of an integrated sensor on a chip, environmental sensor data related to a plurality of environmental conditions;
generating, using a processing core of the integrated sensor, environmental results information as a function of the environmental sensor data related to the plurality of environmental conditions; and
transmitting, under control of the processing core, environmental results information via an interface of the integrated sensor.

37. The method of claim 36 comprising storing at least some of the environmental results information in a non-volatile memory of the integrated sensor.

38. The method of claim 36 wherein the environmental results information comprises an indication of a concentration of a gas and the plurality of environmental conditions comprise one or more of temperature, pressure, illumination, sensor location, sensor position, sensor orientation, and long-term sensor drift.

39. An environmental sensor integrated into a chip, comprising:
means for sensing environmental data related to a plurality of environmental conditions;
means for generating environmental results information as a function of sensed environmental data related to the plurality of environmental conditions; and
means for transmitting environmental results information generated by the means for generating.

40. The system of claim 39 wherein the environmental results information comprises an indication of a concentration of a gas and the plurality of environmental conditions comprise one or more of temperature, pressure, illumination, sensor location, sensor position, sensor orientation, and long-term sensor drift.
